(19) United States
(12) Patent Application Publication
(10) Pub. No.: US 2009/0042189 A1
(43) Pub. Date: Feb. 12, 2009

(54) Increased sensitivity of nucleic acid-based detection of organisms by fractionation of target genomes

(76) Inventor: Frank R. Burns, Philadelphia, PA (US)

Correspondence Address: Rehber Edward F;E I Du Pont De Nemours and Company, Legal Patent Records Center, 4417 Lancaster Pike, Wilmington, DE, 19805, US

(21) Appl. No.: 11/918,610

(22) PCT Filed: Apr. 18, 2006
(86) PCT No.: PCT/US2006/014552

§ 371 Date: Oct. 16, 2007

Related U.S. Application Data
(60) Provisional application No. 60/672,922, filed on Apr. 19, 2005.
(60) Provisional application No. 60/707,886, filed on Aug. 12, 2005.

(51) Int. Cl. C12Q 1/68

(52) U.S. Cl. 435/6

(57) ABSTRACT

A method of analyzing a sample for detection of presence or absence of an organism of interest in the sample comprising (a) obtaining a sample; (b) subjecting the sample to a disruption treatment sufficient to fractionate any nucleic acid sequences present in the sample; and (c) detecting for presence or absence of an organism of interest in the sample. The detecting step (c) may comprise performing primer-directed amplification (preferably polymerase chain reaction) on the sample to produce an amplification result and analyzing the amplification result for an amplification product, wherein presence or absence of the amplification product is indicative of the presence or absence of the organism of interest in the sample.

This application claims the benefit of U.S. Provisional Application No. 60/672,922, filed Apr. 19, 2005, and U.S. Provisional Application No. 60/707,886, filed Aug. 12, 2005, each of which is hereby incorporated by reference in its entirety.

### FIELD OF INVENTION

The field of invention relates to nucleic acid-based detection of organisms, and in particular, to nucleic acid amplification methods and techniques for detecting organisms in a sample.

### BACKGROUND OF INVENTION

Detection of an organism, or group of organisms, can be carried out by detection of specific nucleic acid sequences characteristic of that organism or group of organisms. The presence of the specific nucleic acid sequences can be detected directly by specific hybridization or by amplification using primers that amplify the specific nucleic acid sequences.

In instances where amplification is used as part of the detection method, it is important that at least one copy of the specific nucleic acid sequence to be amplified be present in the sample that is placed in the amplification reaction. When organisms are present in low concentration and/or are in a complex matrix that requires dilution for amplification to proceed, sensitivity can be limited by the need to get at least one copy of the specific nucleic acid sequence into the reaction.

Fungus-specific PCR assays are disclosed in G. Zhou et al., “Development of a fungus-specific PCR assay for detecting low-level fungi in an indoor environment,” Molecular and Cellular Probes (2000) 14, 339-348. This reference discloses a method of bead-beating the sample prior to PCR amplification as the most effective way to effect spore breakage and fungal DNA release. Specifically, a “homogenization-first” procedure is disclosed, in which 0.1 mL of sample is directly added into a 0.5 mL tube containing approximately the same volume of beads. The tube is either fastened on a vortex by rubber bands and vortexed vigorously for 3-5 minutes or put into a “Mini-Bead Beater” and homogenized for 3 minutes, then heated in a boiling water bath for 5-10 minutes to inactivate released nucleases. The reference reports higher PCR amplification efficiency with less inhibition, when using 20% nutrient media and the homogenization-first procedure.

### SUMMARY OF INVENTION

The present invention includes:

A method of analyzing a sample for detection of presence or absence of an organism of interest in the sample, said method comprising: (a) obtaining a sample; (b) subjecting the sample to a disruption treatment sufficient to fractionate any nucleic acid sequences present in the sample; and (c) detecting for presence or absence of an organism of interest in the sample.

A method of analyzing a sample for detection of presence or absence of an organism of interest in the sample, said method comprising: (a) obtaining a sample; (b) preparing the sample by carrying out at least one of the following processes on the sample: (1) enrichment, (2) separation of cells from the sample, (3) cell lysis, and (4) total DNA extraction; (c) subjecting the sample to a disruption treatment sufficient to fractionate any nucleic acid sequences present in the sample; and (d) detecting for presence or absence of an organism of interest in the sample. Preferably, the preparing step comprises carrying out bacterial enrichment and separation of bacterial cells from the sample prior to said step (c); or carrying out bacterial enrichment, separation of bacterial cells from the sample, and cell lysis, prior to said step (c). In another preferred embodiment, in the preparing step, cell lysis is carried out simultaneously with or after said step (c).

A method of analyzing a sample for detection of presence or absence of an organism of interest in the sample, said method comprising: (a) obtaining a sample; (b) enriching the sample; (c) subjecting the enriched sample of step (b) to a disruption treatment sufficient to fractionate any nucleic acid sequences present in the sample; and (d) detecting for presence or absence of an organism of interest in the sample, wherein said step (b) comprises enriching the sample in a vessel, and wherein said step (c) comprises subjecting the enriched sample of said step (b) to said disruption treatment in the same vessel of step (b) without removing the enriched sample therefrom.

Preferably, the disruption treatment comprises adding physical objects (e.g., beads, such as silicon or zirconium) to the sample and agitating the sample containing the physical objects. More preferably, the disruption treatment comprises any physical, mechanical, chemical, or enzymatic disruption treatment.

The detecting step in any of the foregoing methods preferably comprises performing primer-directed amplification (e.g., polymerase chain reaction) on the sample to produce an amplification result and analyzing the amplification result for an amplification product, wherein presence or absence of the amplification product is indicative of the presence or absence of the organism of interest in the sample. Even more preferably, the detecting step comprises: (i) obtaining at least one amplification primer pair set designed to amplify a target sequence located within a repetitive nucleic acid sequence region of a genome of the organism of interest; (ii) performing primer-directed amplification of the sample using the at least one amplification primer pair set to produce an amplification result; and (iii) analyzing the amplification result to detect for presence or absence of amplification product of the target sequence, wherein the presence or absence of the amplification product is indicative, of the presence or absence of the organism of interest in the sample. Preferably, the detection step has at least a ten fold increase in sensitivity in detecting for the organism of interest when compared to detecting without carrying out said step (b).

Preferably, the analyzing of the amplification result is carried out simultaneously with the performing of the primer-directed amplification. The analyzing preferably utilizes 5′-nuclease detection methods.

### BRIEF DESCRIPTION OF DRAWINGS

FIG. 1 shows the process of melting curve analysis. The change in fluorescence of the target DNA is captured during melting. Mathematical analysis of the negative of the change of the log of fluorescence divided by the change in temperature plotted against the temperature results in the graphical peak known as a melting curve.

### DETAILED DESCRIPTION OF PREFERRED EMBODIMENTS

The disclosure of each reference set forth herein is incorporated by reference in its entirety.

### DEFINITIONS

In this disclosure, a number of terms and abbreviations are used. The following definitions are provided.

“Polymerase chain reaction” is abbreviated PCR.

“Polynucleotide”, “nucleic acid sequence”, “nucleotide sequence”, or “nucleic acid fragment” are used interchangeably and is a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

The term “amplification product” refers to nucleic acid fragments produced during a primer-directed amplification reaction. Typical methods of primer-directed amplification include polymerase chain reaction (PCR), ligase chain reaction (LCR) or strand displacement amplification (SDA). If PCR methodology is selected, the replication composition may comprise the components for nucleic acid replication, for example: nucleotide triphosphates, two (or more) primers with appropriate sequences, thermostable polymerase, buffers, solutes and proteins. These reagents and details describing procedures for their use in amplifying nucleic acids are provided in U.S. Pat. No. 4,683,202 (1987, Mullis, et al.) and U.S. Pat. No. 4,683,195 (1986, Mullis, et al.). If LCR methodology is selected, then the nucleic acid replication compositions may comprise, for example: a thermostable ligase (e.g., T. aquaticus ligase), two sets of adjacent oligonucleotides (wherein one member of each set is complementary to each of the target strands), Tris-HCl buffer, KCl, EDTA, NAD, dithiothreitol and salmon sperm DNA. See, for example, Tabor et al., Proc. Acad. Sci. U.S.A., 82:1074 1078 (1985)).

The term “primer” refers to an oligonucleotide (synthetic or occurring naturally), which is capable of acting as a point of initiation of nucleic acid synthesis or replication along a complementary strand when placed under conditions in which synthesis of a complementary stand is catalyzed by a polymerase.

Standard recombinant DNA and molecular cloning techniques used here are well known in the art and are described by Sambrook, J., Fritsch, E. F. and Maniatis, T., Molecular Cloning: A Laboratory Manual, 2nd ed., Cold Spring Harbor Laboratory: Cold Spring Harbor, N.Y. (1989) (hereinafter “Maniatis”); and by Ausubel, F. M. et al., Current Protocols in Molecular Biology, published by Greene Publishing Assoc. and Wiley-Interscience (1987).

Turning now to preferred embodiments:

In a preferred embodiment, a method of analyzing a sample for detection of presence or absence of an organism of interest in the sample comprises: obtaining a sample; subjecting the sample to a disruption treatment sufficient to fractionate any nucleic acid sequences present in the sample; and detecting for presence or absence of an organism of interest in the sample.

In another preferred embodiment, a method for increasing sensitivity of detection of an organism of interest in a sample comprises: obtaining a sample; subjecting the sample to a disruption treatment sufficient to fractionate any nucleic acid sequences present in the sample; and detecting for presence or absence of an organism of interest in the sample, wherein sensitivity in said detecting step is increased by preferably at least 10 fold, more preferably at least any integer from 11-100 fold, more preferably from 10 fold to any integer from 11-100 fold, more preferably from any integer from 10-99 fold to 100 fold, or more preferably from any integer from 10-100 fold to any integer between 100-10 fold, when compared to not carrying out said step of subjecting the sample to a disruption treatment.

Preferably, the detecting step comprises performing primer-directed amplification (preferably polymerase chain reaction) on the sample to produce an amplification result and analyzing the amplification result for an amplification product, wherein presence or absence of the amplification product is indicative of the presence or absence of the organism of interest in the sample. Even more preferably, the detecting step comprises: obtaining at least one amplification primer pair set designed to amplify a target sequence located within a repetitive nucleic acid sequence region of a genome of the organism of interest; performing primer-directed amplification of the sample using the at least one amplification primer pair set to produce an amplification result; and analyzing the amplification result to detect for presence or absence of amplification product of the target sequence, wherein the presence or absence of the amplification product is indicative of the presence or absence of the organism of interest in the sample.

More preferably, the analyzing of the amplification result is carried out simultaneously with the performing of the primer-directed amplification (e.g., polymerase chain reaction). Even more preferably, the analyzing of the amplification product utilizes 5′-nuclease detection methods.

In another preferred embodiment, in any of the methods of the present invention, a step of enrichment is carried out on the sample prior to the step of subjecting the sample to a disruption treatment. The enrichment of the sample is carried out in a vessel, and preferably the step of subjecting the sample to a disruption treatment is carried out on the enriched sample in the same vessel in which the sample has been enriched (i.e., both enrichment and disruption treatment may be carried out on the sample in the same vessel). Preferably, the vessel is a sample tube, but the vessel may also be any suitable vessel or container known in the art, for example, test tubes, beakers, flasks, jars, vials, ampules, or microfuge tubes.

Preferred Samples and Organisms of Interest

Preferably, the sample is from an animal, environmental or food source suspected of contamination by the organism of interest.

The organism of interest may be any living thing. Preferably, the organism of interest is a microorganism. Specific organisms of interest which may be detected by methods of the present invention preferably include bacteria and fungi.

Particularly preferred organisms of interest are fungal organisms. Particularly preferred is the fungi Sacchromyces cervasiae.

The methods of the present invention are believed to be particularly advantageous in increasing the sensitivity of detection of fungal organisms in a sample.

Without being bound to a particular theory or mechanism, it is believed that increased sensitivity is achieved particularly with fungal organisms, since fungal organism genomes typically contain 100-200 copies of their ribosomal ribonucleic acid (rRNA) genes tandemly arrayed in repeating units of approximately 10,000 base pairs each.

In a typical primer-directed nucleic acid amplification strategy, the equivalent of 4 μL of the material being tested is placed in the amplification reaction. If there are 20 haploid fungi per mL, then on average one chromosome, carrying a target amplification region in the rRNA gene, is distributed per 50 μL (=1 mL/20). Sampling 4 μL is therefore unlikely to result in a target amplification region being present when primer-directed nucleic acid amplification is carried out on the 4 μL sample.

However, by subjecting a sample containing a fungal organism to a disruption treatment (explained below) in accordance with the present invention, then in a 1 mL sample containing 20 haploid fungi, if the disruption treatment is carried out to fractionate nucleic acid sequences from the fungal genome into approximately 10,000 base pairs, the 1 mL sample now has on average 2,000 to 4,000 targets (from the 100-200 rRNA genes) per mL, or on average 8-16 targets per 4 μL sample, thereby increasing the sensitivity in detection particularly when primer-directed nucleic acid amplification is employed.

Thus, even more preferably, in another preferred embodiment, methods of the present invention may be employed to detect or to increase sensitivity in the detection of any organism of interest having a genome in which repetitive nucleic acid sequences exist and serve as target regions for primer-directed amplification. Sensitivity is increased by preferably at least 10 fold, more preferably at least any integer from 11-100 fold, more preferably from 10 fold to any integer from 11-100 fold, more preferably from any integer from 10-99 fold to 100 fold, or more preferably from any integer from 10-100 fold to any integer from 100-10 fold, when compared to not carrying out a disruption treatment in accordance with the present invention.

Preferred Disruption Treatments

Once a sample is obtained, the next step is to subject the sample to a disruption treatment sufficient to fractionate any nucleic acid sequences present in the sample. Preferably, the nucleic acid sequences are from the genome of the organism of interest for which the sample is being analyzed.

The disruption treatment may comprise any method or protocol for fractionating nucleic acid sequences in the sample, and in particular, for fractionating nucleic acid sequences from the genome of the organism of interest for which the sample is being analyzed. The disruption treatment may employ physical, chemical, or enzymatic approaches, and is not limited to these particular approaches. Any nucleic acid fractionation approach known to or readily ascertainable by one of ordinary skill in the art may be utilized and is included within the present invention. The disruption treatment is carried out for a time period sufficient to fractionate any nucleic acid sequences present in the sample, and one of ordinary skill can readily determine the length of this time period depending on the sample and the organism of interest for which the sample is being tested.

Preferred physical disruption treatments include the use of physical objects, such as beads, in combination with agitation of a sample. The agitation conditions should be sufficient to produce shearing forces that break the DNA into smaller fragments. Other physical disruption treatments include sonication, nebulization, or passage of DNA-containing solution through a narrow orifice.

Preferred chemical disruption treatments include treatment with sodium hydroxide, treatment with dimethylsulfate followed by peperidine, treatment with peperidine formate followed by treatment with piperidince, or treatment with hydrazine followed by piperidine.

Preferred enzymatic disruption treatments include digestion with nonspecific nucleases such as DNase 1 or digestion with restriction endonucleases such as EcoR1.

In a particularly preferred embodiment, the disruption treatment comprises adding physical objects to the sample and agitating the sample containing the physical objects. The agitation may be carried out for a time period sufficient to fractionate any nucleic acid sequences present in the sample, and one of ordinary skill can readily determine the length of this time period depending on the sample and the organism of interest for which the sample is being tested. The agitation is carried out preferably for at least 6 minutes, more preferably for at least any integer from 6-20 minutes, more preferably from 6 minutes to any integer from 6-20 minutes, more preferably from any integer from 6-19 minutes to 20 minutes, or more preferably from any integer from 6-20 minutes to any integer from 20-6 minutes. In a particularly preferred embodiment, agitation is carried out for at least 15 minutes. In another preferred embodiment, agitation is carried out for 15-20 minutes. A particularly preferred apparatus for carrying out the agitation is a Disruptor Genie® (available from Scientific Industries, Inc., Bohemia, N.Y.). The physical objects preferably comprise beads, and even more preferably silicon or zirconium beads. The physical objects may also comprise glass beads.

The methods according to the instant invention may be used directly with any suitable clinical or environmental samples, without any need for sample preparation. However, in order to achieve higher sensitivity, and in situations where time is not a limiting factor, it is preferred that the samples be pre-treated.

In another preferred embodiment, therefore, prior to detecting for the target organism of interest in the sample, a step of preparing the sample may be carried out. Preferably, this preparing step may comprise at least one of the following processes: enrichment (e.g., of the target organism) in growth media, separation of cells (e.g., of the target organism) from the sample, cell lysis, and DNA extraction. More preferably, this preparing step comprises carrying out enrichment and separation prior to subjecting the sample to a disruption treatment. Even more preferably, this preparing step comprises carrying out enrichment, separation, and cell lysis, prior to subjecting the sample to a disruption treatment. In another preferred embodiment, cell lysis is carried out simultaneously with or after the disruption treatment. Examples of sample preparation steps carried out in conjunction with methods of the present invention are set forth in Example 1 below.

Typical enrichment procedures employ media that will enhance the growth and health of the target organisms and also inhibit the growth of any background or non-target microorganisms present. Selective media have been developed for a variety of organisms, and one of skill in the art will know to select a medium appropriate for the particular organism to be enriched. A general discussion and recipes of non-selective media are described in the FDA Bacteriological Analytical Manual. (1998) published and distributed by the Association of Analytical Chemists, Suite 400, 2200 Wilson Blvd, Arlington, Va. 22201-3301.

After growth, a sample of the complex mixtures is removed for further analysis. This sampling procedure may be accomplished by a variety of means well known to those skilled in the art.

In a preferred embodiment, 20 μL of the enrichment culture is removed and added to 200 μL of lysis solution containing protease. The lysis solution is heated at 37° C. for 20 min followed by protease inactivation at 95° C. for 10 min as described in the BAX® System User's Guide, Qualicon, Inc., Wilmington, Del.

Alternatively, in a more preferred embodiment, in any of the methods of the present invention, a step of enrichment is carried out on the sample prior to the step of subjecting the sample to a disruption treatment, wherein the enrichment of the sample is carried out in a vessel, and the step of subjecting the sample to a disruption treatment is carried out on the enriched sample in the same vessel in which the sample had been enriched (i.e., both enrichment and the disruption treatment is carried out on the sample in the same vessel). Preferably, the vessel is a sample tube, but the vessel may also be any suitable vessel or container known in the art, for example, a test tube, beaker, flask, jar, vial, ampule, or microfuge tube.

Preferred Detection and Analysis

A variety of nucleic acid-based detection methods may be employed for the detection of the target organism(s) of interest. A variety of primer-directed nucleic acid amplification methods are known in the art including thermal cycling methods (e.g., PCR, RT-PCR, and LCR), as well as isothermal methods and strand displacement amplification (SDA), nucleic acid sequence-based amplification (NASBA), and self-sustained sequence replication (3SR) and ‘Q replicase amplification’.

The preferred method is PCR.

Preferably, in any method of the present invention, the detection step utilizes an amplification primer pair set designed to amplify a target sequence located within a repetitive nucleic acid sequence region of a genome of the organism of interest in the sample to be tested.

Any suitable nucleic acid replication composition (“replication composition”) in any format can be used.

A typical replication composition for PCR amplification may comprise, for example, dATP, dCTP, dGTP, dTTP, target specific primers and a suitable polymerase.

If the replication composition is in liquid form, suitable buffers known in the art may be used (Sambrook, J. et al. 1989, Molecular Cloning: A Laboratory Manual, Second Edition, Cold Spring Harbor Laboratory Press).

Alternatively, if the replication composition is contained in a tablet form, then typical tabletization reagents may be included such as stabilizers and binding agents. Preferred tabletization technology is set forth in U.S. Pat. Nos. 4,762,857 and 4,678,812, each of which is hereby incorporated by reference in its entirety.

In another preferred embodiment, a replication composition contains an internal positive control. The advantages of an internal positive control contained within a PCR reaction have been previously described (U.S. Pat. No. 6,312,930 and PCT Application No. WO 97/11197, each of which is hereby incorporated by reference in its entirety) and include: (i) the control may be amplified using a single primer; (ii) the amount of the control amplification product is independent of any target DNA or RNA contained in the sample; (iii) the control DNA can be tableted with other amplification reagents for ease of use and high degree of reproducibility in both manual and automated test procedures; (iv) the control can be used with homogeneous detection, i.e., without separation of product DNA from reactants; and (v) the internal control has a melting profile that is distinct from other potential amplification products in the reaction

Control DNA will be of appropriate size and base composition to permit amplification in a primer-directed amplification reaction. The control DNA sequence may be obtained from the genome of the organism of interest, or from another source, but must be reproducibly amplified under the same conditions that permit the amplification of the target amplification product.

The control reaction is useful to validate the amplification reaction. Amplification of the control DNA occurs within the same reaction tube as the sample that is being tested, and therefore indicates a successful amplification reaction when samples are target negative, i.e. no target amplification product is produced. In order to achieve significant validation of the amplification reaction a suitable number of copies of the control DNA must be included in each amplification reaction.

In some instances it may be useful to include an additional negative control replication composition. The negative control replication composition will contain the same reagents as the replication composition but without the polymerase. The primary function of such a control is to monitor spurious background fluorescence in a homogeneous format when the method employs a fluorescent means of detection. Replication compositions may be modified depending on whether they are designed to be used to amplify target DNA or the control DNA.

Replication compositions that will amplify the target DNA (test replication compositions) may include (i) a polymerase (generally thermostable), (ii) a primer pair capable of hybridizing to the target DNA and (iii) buffers for the amplification reaction to proceed.

Replication compositions that will amplify the control DNA (positive control, or positive replication composition) may include (i) a polymerase (generally thermostable) (ii) the control DNA; (iii) at least one primer capable of hybridizing to the control DNA; and (iv) buffers for the amplification reaction to proceed.

A skilled person will understand that any generally acceptable PCR conditions may be used for successfully detecting the target organism of interest, and depending on the sample to be tested and other laboratory conditions, routine optimization for the PCR conditions may be needed to achieve optimal sensitivity and specificity. Optimally, they achieve PCR amplification products from all of the intended specific targets while giving no PCR product for other, non-target species.

In a preferred embodiment, the following reagents and cycling conditions may be used. Fifty microliters of lysate added to a PCR tube containing one BAX® reagent tablet (manufactured by Qualicon, Inc., Wilmington, Del.), the tablet containing Taq DNA polymerase, deoxynucleotides, SYBR® Green (Molecular Probes, Eugene, Oreg.), buffer components, and primers to achieve a final concentration in the PCR of 0.150 micromoles for each primer. Preferred PCR cycling conditions: 94° C., 2 minutes initial DNA denaturation, followed by 38 cycles of denaturation at 94° C., 15 seconds, annealing at 56° C. for 45 seconds and extension at 70° C. for 2 minutes, followed by a single final hold at 70° C.

Primer-directed amplification results can be analyzed using various methods to detect for the presence or absence of an amplification product.

Homogenous detection refers to a preferred method for the detection of amplification products where no separation (such as by gel electrophoresis) of amplification products from template or primers is necessary. Homogeneous detection is typically accomplished by measuring the level of fluorescence of the reaction mixture in the presence of a fluorescent dye.

In a preferred embodiment, DNA melting curve analysis is used to carry out homogenous detection, particularly with the BAX® System hardware and reagent tablets from Qualicon Inc. The details of the system are given in U.S. Pat. No. 6,312,930 and PCT Publication Nos. WO 97/11197 and WO 00/66777, each of which is hereby incorporated by reference in its entirety.

Melting curve analysis detects and quantifies double stranded nucleic acid molecule (“dsDNA” or “target”) by monitoring the fluorescence of the target amplification product (“target amplicon”) during each amplification cycle at selected time points.

As is well known to the skilled artisan, the two strands of a dsDNA separate or melt, when the temperature is higher than its melting temperature. Melting of a dsDNA molecule is a process, and under a given solution condition, melting starts at a temperature (designated T_{MS} hereinafter), and completes at another temperature (designated T_{ME} hereinafter). The familiar term, Tm, designates the temperature at which melting is 50% complete.

A typical PCR cycle involves a denaturing phase where the target dsDNA is melted, a primer annealing phase where the temperature optimal for the primers to bind to the now-single-stranded target, and a chain elongation phase (at a temperature T_{E}) where the temperature is optimal for DNA polymerase to function.

According to the present invention, T_{MS} should be higher than T_{E}, and T_{ME} should be lower (often substantially lower) than the temperature at which the DNA polymerase is heat-inactivated. Melting characteristics are effected by the intrinsic properties of a given dsDNA molecule, such as deoxynucleotide composition and the length of the dsDNA.

Intercalating dyes will bind to double stranded DNA. The dye/dsDNA complex will fluoresce when exposed to the appropriate excitation wavelength of light, which is dye dependent, and the intensity of the fluorescence may be proportionate to concentration of the dsDNA. Methods taking advantage of the use of DNA intercalating dyes to detect and quantify dsDNA are known in the art. Many dyes are known and used in the art for these purposes. The instant methods also take advantage of such relationship.

An example of such dyes includes intercalating dyes. Examples of such dyes include, but are not limited to, SYBR Green I®, ethidium bromide, propidium iodide, TOTO® 1 {Quinolinium, 1 1′[1,3 propanediylbis [(dimethyliminio)-3,1-propanediyl]]bis[4-[(3-methyl-2(3H)-benzothiazolylidene)methyl]]-, tetraiodide}, and YoPro® {Quinolinium, 4[(3-methyl-2(3H)-benzoxazolylidene)methyl]-1-[3-(trimethylammonio) [symbol] propyl],diiodide}. Most preferred for the instant invention is a non-asymmetrical cyanide dye such as SYBR Green-I®, manufactured by Molecular Probes, Inc. (Eugene, Oreg.).

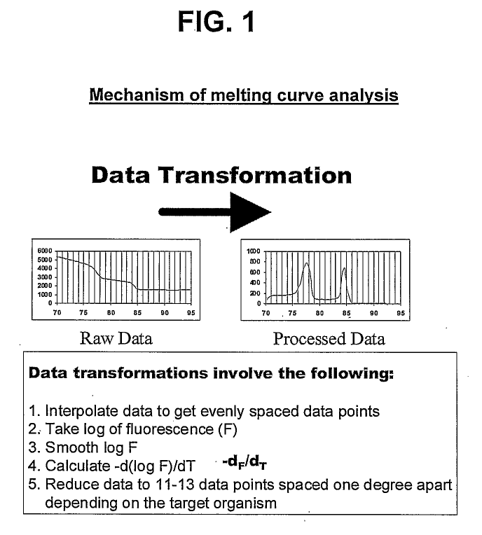

Melting curve analysis is achieved by monitoring the change in fluorescence while the temperature is increased. When the temperature reaches the T_{MS} specific for the target amplicon, the dsDNA begins to denature. When the dsDNA denatures, the intercalating dye dissociates from the DNA and fluorescence decreases. Mathematical analysis of the negative of the change of the log of fluorescence divided by the change in temperature plotted against the temperature results in the graphical peak known as a melting curve (See FIG. 1, which illustrates melting curve analysis in general).

The data transformation process shown in FIG. 1 involves the following:

1. Interpolate data to get evenly spaced data points

2. Take a log of the fluorescence (F)

3. Smooth log F

4. Calculate -d(log F)/dT

5. Reduce data to 11-13 data points spaced one degree apart (depending on the target organism).

The instant homogenous detection method can be used to detect and quantify target dsDNAs, from which the presence and level of target organisms can be determined. This method is very specific and sensitive. The fewest number of target dsDNA detectable is between one and 10 under typical reaction conditions and volumes.

Homogenous detection may be employed to carry out “real-time” primer-directed nucleic acid amplifications, using primer pairs of the instant invention (e.g., “real-time” PCR and “real-time” RT-PCR). Preferred “real-time” methods are set forth, for example, in U.S. Pat. Nos. 6,171,785 and 5,994,056, each of which is hereby incorporated by reference in its entirety.

Another detection method is the 5′ nuclease detection method, as set forth, for example, in U.S. Pat. Nos. 5,804,375, 5,538,848, 5,487,972, and 5,210,015, each of which is hereby incorporated by reference in its entirety.

A variety of other PCR detection methods are known in the art including standard non-denaturing gel electrophoresis (e.g., acrylamide or agarose), denaturing gradient gel electrophoresis, and temperature gradient gel electrophoresis. Standard non-denaturing gel electrophoresis is a simple and quick method of PCR detection, but may not be suitable for all applications.

Denaturing Gradient Gel Electrophoresis (DGGE) is a separation method that detects differences in the denaturing behavior of small DNA fragments (200-700 bp). The principle of the separation is based on both fragment length and nucleotide sequence. In fragments that are the same length, a difference as little as one base pair can be detected. This is in contrast to non-denaturing gel electrophoresis, where DNA fragments are separated only by size. This limitation of non-denaturing gel electrophoresis results because the difference in charge density between DNA molecules is near neutral and plays little role in their separation. As the size of the DNA fragment increases, its velocity through the gel decreases.

DGGE is primarily used to separate DNA fragments of the same size based on their denaturing profiles and sequence. Using DGGE, two strands of a DNA molecule separate, or melt, when heat or a chemical denaturant is applied. The denaturation of a DNA duplex is influenced by two factors: 1) the hydrogen bonds formed between complimentary base pairs (since GC rich regions melt at higher denaturing conditions than regions that are AT rich); and 2) the attraction between neighboring bases of the same strand, or “stacking”. Consequently, a DNA molecule may have several melting domains with each of their individual characteristic denaturing conditions determined by their nucleotide sequence. DGGE exploits the fact that otherwise identical DNA molecules having the same length and DNA sequence, with the exception of only one nucleotide within a specific denaturing domain, will denature at different temperatures or Tm. Thus, when the double-stranded (ds) DNA fragment is electrophoresed through a gradient of increasing chemical denaturant it begins to denature and undergoes both a conformational and mobility change. The dsDNA fragment will travel faster than a denatured single-stranded (ss) DNA fragment, since the branched structure of the single-stranded moiety of the molecule becomes entangled in the gel matrix. As the denaturing environment increases, the ds DNA fragment will completely dissociate and mobility of the molecule through the gel is retarded at the denaturant concentration at which the particular low denaturing domains of the DNA strand dissociate. In practice, the electrophoresis is conducted at a constant temperature (around 60° C.) and chemical denaturants are used at concentrations that will result in 100% of the DNA molecules being denatured (i.e., 40% formamide and 7M urea). This variable denaturing gradient is created using a gradient maker, such that the composition of each DGGE gel gradually changes from 0% denaturant up to 100% denaturant. Of course, gradients containing a reduced range of denaturant (e.g., 35% to 60%) may also be poured for increased separation of DNA.

The principle used in DGGE can also be applied to a second method that uses a temperature gradient instead of a chemical denaturant gradient. This method is known as Temperature Gradient Gel Electrophoresis (TGGE). This method makes use of a temperature gradient to induce the conformational change of dsDNA to ssDNA to separate fragments of equal size with different sequences. As in DGGE, DNA fragments with different nucleotide sequences will become immobile at different positions in the gel. Variations in primer design can be used to advantage in increasing the usefulness of DGGE for characterization and identification of the PCR products. These methods and principles of using primer design variations are described in PCR Technology Principles and Applications, Henry A. Erlich Ed., M. Stockton Press, NY, pages 71 to 88 (1988).

### EXAMPLE

The present invention is further exemplified in the following Example. It should be understood that this Example, while indicating preferred embodiments of the invention, is given by way of illustration only.

### Example 1

An overnight culture of the fungi Sacchromyces cervasiae (S. cervasiae) in potato dextrose broth was diluted serially by 5 fold in a solution of 1% tryptone with 2 mM EDTA (ethylene diamine tetracetate). Each dilution was simultaneously analyzed in accordance with each of the following three methods:
1. The dilution was plated on potato dextrose agar to determine the concentration of S. cervasiae.
2. A portion of the sample was subjected to a chemical cell lysis followed by PCR amplification using a BAX® System yeast and mold assay detection kit (available from DuPont Qualicon, Wilmington, Del.), which uses fungal specific primers that amplify a portion of the 18s ribosomal RNA gene and detect the amplicon, in a BAX® System instrument (available from DuPont Qualicon, Wilmington, Del.).
3. A portion of the sample was placed in a tube with 0.5 mM silica-zirconium beads, agitated for 15 minutes in a Disruptor Genie®, and then an aliquot was subjected to a chemical cell lysis followed by PCR amplification in a reaction using a BAX® System yeast and mold assay detection kit, which uses fungal specific primers that amplify a portion of the 18s ribosomal RNA gene and detect the amplicon, in a BAX® System instrument.

Samples were run with different amounts of colony forming units of S. cervasiae as the starting input in accordance with methods 2 and 3 above. The results are displayed below in Table 1. As shown below, method 3 increases the sensitivity of detection at levels below 2 CFU.

TABLE 1

| | BAX ® Assay Result- | BAX ® Assay Result- |
|---|---|---|
| Input CFU/PCR | Bead Agitated | No Beads |
| reaction | (method 3 above) | (method 2 above) |
| 50 | + | + |
| 10 | + | + |
| 2 | + | + |
| 0.4 | + | − |
| 0.08 | + | − |

What is claimed is:

1. A method of analyzing a sample for detection of presence or absence of an organism of interest in the sample, said method comprising:
(a) obtaining a sample;
(b) subjecting the sample to a disruption treatment sufficient to fractionate any nucleic acid sequences present in the sample; and
(c) detecting for presence or absence of the organism of interest in the sample.

2. The method of claim 1, wherein in said step (b), the disruption treatment comprises adding physical objects to the sample and agitating the sample containing the physical objects.

3. The method claim 2, wherein said physical objects comprise beads.

4. The method of claim 3, wherein said beads comprise silicon or zirconium.

5. The method of claim 1, wherein in said step (b), the disruption treatment comprises a physical, chemical, or enzymatic disruption treatment.

6. The method of claim 1, further comprising, prior to said step (c), a step of preparing the sample by carrying out at least one of the following processes on the sample: (1) enrichment, (2) separation of cells from the sample, (3) cell lysis, and (4) total DNA extraction.

7. The method of claim 6, wherein said preparing step comprises carrying out bacterial enrichment and separation of cells from the sample prior to said step (b).

8. The method of claim 6, wherein said preparing step comprises carrying out bacterial enrichment, separation of cells from the sample, and cell lysis, prior to said step (b).

9. The method of claim 6, wherein in said preparing step said cell lysis is carried out simultaneously with or after said step (b).

10. The method claim 1, wherein said detecting step (c) comprises performing primer-directed amplification on the sample to produce an amplification result and analyzing the amplification result for an amplification product, wherein presence or absence of the amplification product is indicative of the presence or absence of the organism of interest in the sample.

11. The method of claim 1, wherein said detecting step (c) comprises:
(i) obtaining at least one amplification primer pair set designed to amplify a target sequence located within a repetitive nucleic acid sequence region of a genome of the organism of interest;
(ii) performing primer-directed amplification of the sample using the at least one amplification primer pair set to produce an amplification result; and
(iii) analyzing the amplification result of said step (c)(ii) to detect for presence or absence of amplification product of the target sequence, wherein the presence or absence of the amplification product is indicative of the presence or absence of the organism of interest in the sample.

12. The method of claim 11, wherein in said step (c) the primer-directed amplification is polymerase chain reaction.

13. The method of claim 12, wherein said analyzing of the amplification result is carried out simultaneously with said performing of the polymerase chain reaction.

14. The method of claim 12, wherein in said step (c) said analyzing of the amplification result utilizes 5′-nuclease detection methods.

15. The method of claim 11, wherein said step (c) has at least a ten fold increase in sensitivity in detecting for the organism of interest when compared to detecting without carrying out said step (b).

16. A method of analyzing a sample for detection of presence or absence of an organism of interest in the sample, said method comprising:
(a) obtaining a sample;
(b) enriching the sample;
(c) subjecting the enriched sample of step (b) to a disruption treatment sufficient to fractionate any nucleic acid sequences present in the sample; and
(d) detecting for presence or absence of the organism of interest in the sample, said detecting step comprising:
(i) obtaining at least one amplification primer pair set designed to amplify a target sequence located within a repetitive nucleic acid sequence region of a genome of the organism of interest;
(ii) performing primer-directed amplification of the sample using the at least one amplification primer pair set to produce an amplification result; and
(iii) analyzing the amplification result of said step (d)(ii) to detect for presence or absence of amplification product of the target sequence, wherein the presence or absence of the amplification product is indicative of the presence or absence of the organism of interest in the sample.

17. The method of claim 16, wherein said step (b) comprises enriching the sample in a vessel, and wherein said step (c) comprises subjecting the enriched sample of said step (b) to said disruption treatment in the same vessel of step (b) without removing the enriched sample therefrom.
